(19) United States
(12) Patent Application Publication
(10) Pub. No.: US 2013/0253391 A1
(43) Pub. Date: Sep. 26, 2013

(54) FOOT CARE APPARATUS

(71) Applicant: SACHIN ANIL BRAHMBHATT, Jacksonville, FL (US)

(72) Inventor: SACHIN ANIL BRAHMBHATT, Jacksonville, FL (US)

(21) Appl. No.: 13/774,922

(22) Filed: Feb. 22, 2013

Related U.S. Application Data
(60) Provisional application No. 61/613,931, filed on Mar. 21, 2012.

(51) Int. Cl. A61B 17/50; A61H 99/00

(52) U.S. Cl. CPC A61B 17/50; A61H 99/00
USPC 601/154; 606/131

(57) ABSTRACT

An apparatus allows a person with limited mobility to treat a top, bottom, or side surface of his foot with a lotion, abrade a callous, massage his foot, or clean his foot. A mounting head is fixed to a handle such that the mounting head and a tool for treating a foot of a person fixed to the mounting head can rotate to a wide range of orientations to facilitate access by the tool to top, bottom, side and heel surfaces of a person's foot. A variety of tools may be attached to the mounting head in a removable and replaceable manner.

### PRIORITY CLAIM

This application claims priority to U.S. Provisional Patent Application Ser. No. 61/613,931, entitled “ Foot care apparatus ” filed 21 Mar. 2012, the disclosure of which is incorporated, in its entirety, herein.

### FIELD OF THE INVENTION

The present invention relates to a foot care apparatus having a replaceable tool for treating a callus or for applying lotion, or cleaning a foot of a person.

1. Background of the Invention

Many people have difficulty accessing surfaces of their feet to clean or maintain their feet to promote healthy feet. The result is that such persons must either obtain help from another person to wash and maintain their feet, or must seek devices that facilitate access to their feet. While some devices have been disclosed for aiding these people, some of those devices require a person to reach their feet with their hands which is impossible for many persons to do for a variety of reasons such as arthritis, obesity, chronic back pain, or simply any injury. While dry skin is not a dangerous condition, it can become painful, and if the cracking starts to bleed, it can lead to infection which is an especially serious problem for anyone with a chronic disease such as diabetes, or a lowered immune system due to age or illness. Infections can lead to ulcerations and if not treated it can lead to amputation. Calluses can also be painful, crack, and become infected. Other prior art foot care devices do not facilitate access to all of the main surfaces of the foot, namely the top, sides, bottom and heel. There is a need for a foot care device that can be used by a person of limited agility to apply lotion or treat a callus on the main surfaces of his or her feet.

2. Discussion of the Prior Art

U.S. Pat. No. 3,531,814 A discloses a device that enables a person to maintain an upright position instead of having to bend for cleaning the web spaces between his or her toes. However, this device is not designed to facilitate cleaning all of the major surfaces of a person's feet.

U.S. Pat. No. 4,961,733 A discloses a cleaning and chemical applicator element, attached to a handle, for cleaning and application of chemicals between the toes of a person's foot. The device is provided with a handle that is “preferably the same length as the distance between a typical person's knee and foot”. However, this device is not designed to facilitate cleaning and the application of chemicals to all of the major surfaces of a person's feet.

U.S. Pat. No. 5,163,200 A discloses another approach to foot cleaning for a person of limited agility by providing a foot scrubbing apparatus that has a compressible housing formed with a rigid floor having a matrix of suction cups thereon. The suction cups are used to fix the device to a floor of a shower stall or a wall of a bathtub. A person can then move his feet on the device to clean his feet, but obviously not all of the major surfaces of a person's feet can be cleaned with such a device.

U.S. Pat. No. 5,560,068 A discloses a shower brush for cleaning various portions of a person's foot. The disclosed shower brush has no provision for applying a lotion to a person's foot and may not be functional for a person having limited agility.

U.S. Pat. No. 5,673,455 A discloses an applicator device for moving a contact surface against a person's back that includes an elongated handle and at one end a cap formed with structure for brushing, massaging or scratching a person's back using a variety of replaceable pads. If this device were used on a person's feet the rigid attachment of the cap to the handle could restrict the contact of the pad with the entire area of the major surfaces of the foot in any convenient manner.

U.S. Pat. No. 7,572,238 B2 discloses an apparatus for abrading human skin and applying a composition with a short handle and at one end has an applicator comprising a porous mass coupled to a head portion and a sonic wave generator propagates sonic waves through the applicator. The applicator is taught to be attached rigidly to the handle which could restrict the contact of the pad with the entire area of the major surfaces of the foot in any convenient manner.

U.S. Pat. No. D628,391 S discloses an ornamental design for a foot scrubbing brush that has a curved portion disposed at one end of a handle with the brush located at an end of the curved portion that is distal from the handle. There is no teaching of suggestion that this design may be used to apply lotion to a person's foot or that a person of limited agility could reach the top, bottom and side surfaces of his or her foot with this device.

### SUMMARY OF THE INVENTION

There is provided in accordance with the present invention a foot care apparatus that allows a person with limited mobility to treat a top, bottom, or side surface of his foot with a lotion, abrade a callous or clean his foot. The new apparatus has a handle with a frame having a base portion adjacent to a first end of the handle for attaching a mounting head to the apparatus. The frame has two spaced apart legs that originate at the base portion wherein each of the legs follows a curved path and the legs are spaced apart from one another a constant distance along their entire lengths. A mounting head is attached to the frame with a portion of the mounting head disposed between the legs of the frame. The mounting head fixed to the frame in a manner that allows the mounting head to rotate on an axis that extends between the legs of the frame. A tool for treating a foot of a person is fixed to the mounting head. The tool may be attached to the mounting head in a removable and replaceable manner. The tool may be for example a pad for applying a lotion to the foot of a person, or an abrasive tool for treating a callous, or a brush that may be used for cleaning a person's foot, or a cylinder-like foam member for cleaning between a person's toes.

### BRIEF DESCRIPTION OF THE DRAWINGS

FIG. 1 is a perspective view of a foot care apparatus of the present invention.

FIG. 2 is another perspective view of the foot care apparatus of the present invention.

FIG. 3 is a front elevation view of the foot care apparatus of the present invention.

FIG. 4 is a side elevation view of the foot care apparatus of the present invention.

FIG. 5 is a bottom view I of the foot care apparatus of the present invention looking upwards in the direction indicated by arrow A in FIG. 3.

FIG. 6 is a top view of the foot care apparatus of the present invention looking downwards in the direction indicated by arrow B in FIG. 3.

FIG. 7 is an exploded perspective view of a subassembly of a mounting head and a tool for treating a foot of a person.

FIG. 8 is a fragmentary perspective view of the foot care apparatus of the present invention equipped with a tool that is an abrasive member.

FIG. 9 is a fragmentary perspective view of the foot care apparatus of the present invention equipped with a tool that is a brush.

FIG. 10 is a fragmentary perspective view of the foot care apparatus of the present invention equipped with a tool that is a massage head.

FIG. 11 is a fragmentary perspective view of the foot care apparatus of the present invention equipped with a tool that may be used for cleaning between a person's toes.

FIG. 12 is a fragmentary perspective view of another embodiment of a foot care apparatus of the present invention wherein the spaced apart legs of the frame used for attaching a mounting head to the apparatus are straight instead of curved as in the prior figures.

### DETAILED DESCRIPTION OF THE INVENTION

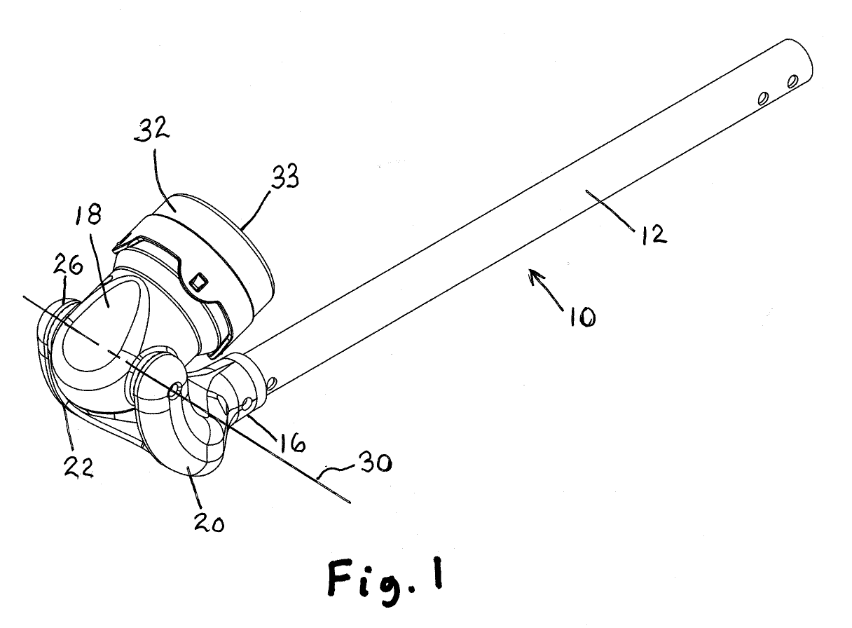

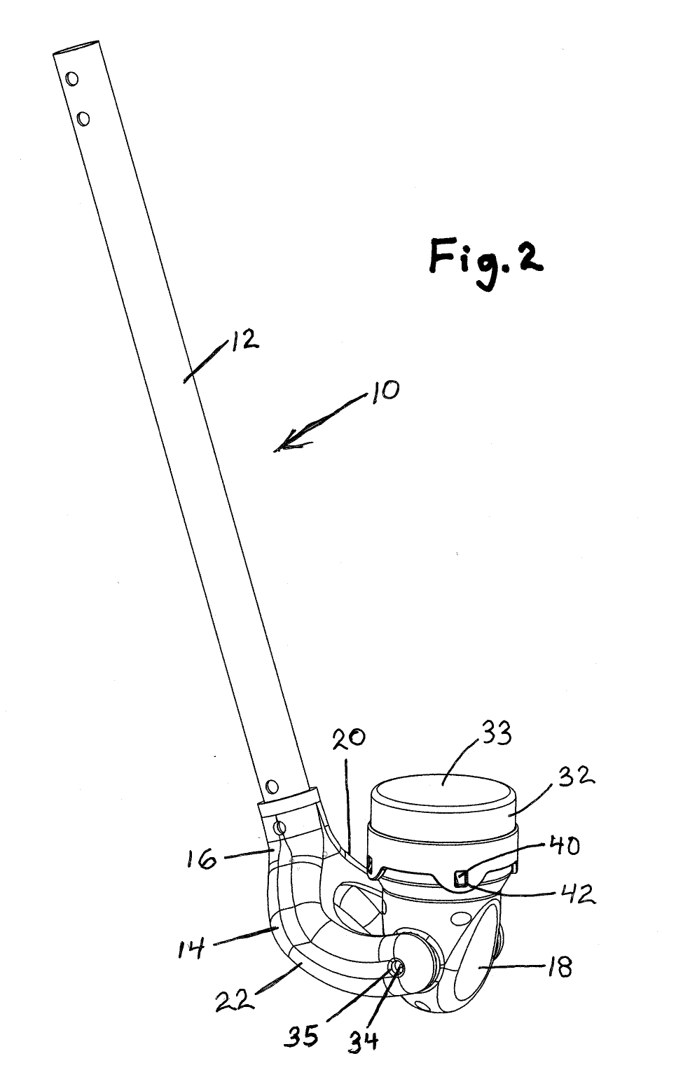

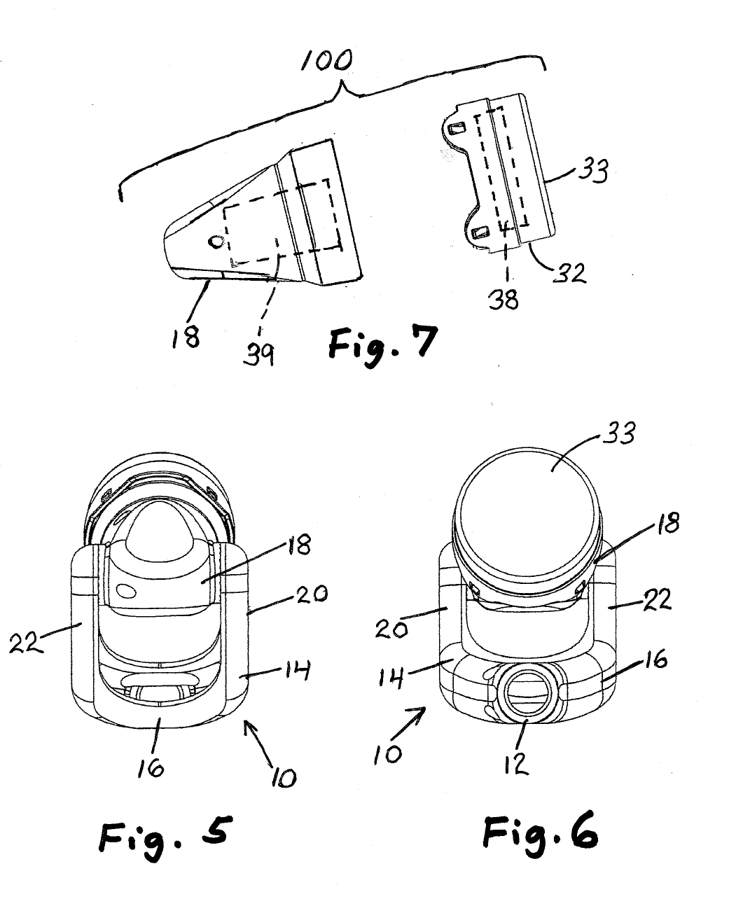

Referring to FIGS. 1-6 a foot care apparatus 10 of the present invention has a handle 12 having a length that is greater than a width or diameter of the handle. Preferably the handle has a length that is variable to accommodate use of the apparatus by persons of various sizes. The handle may be made of any suitable material selected in accordance with good engineering practices, such as a polymer or a metal. It is understood that the length of the handle may be varied using a number of known structures, such as a telescoping arrangement.

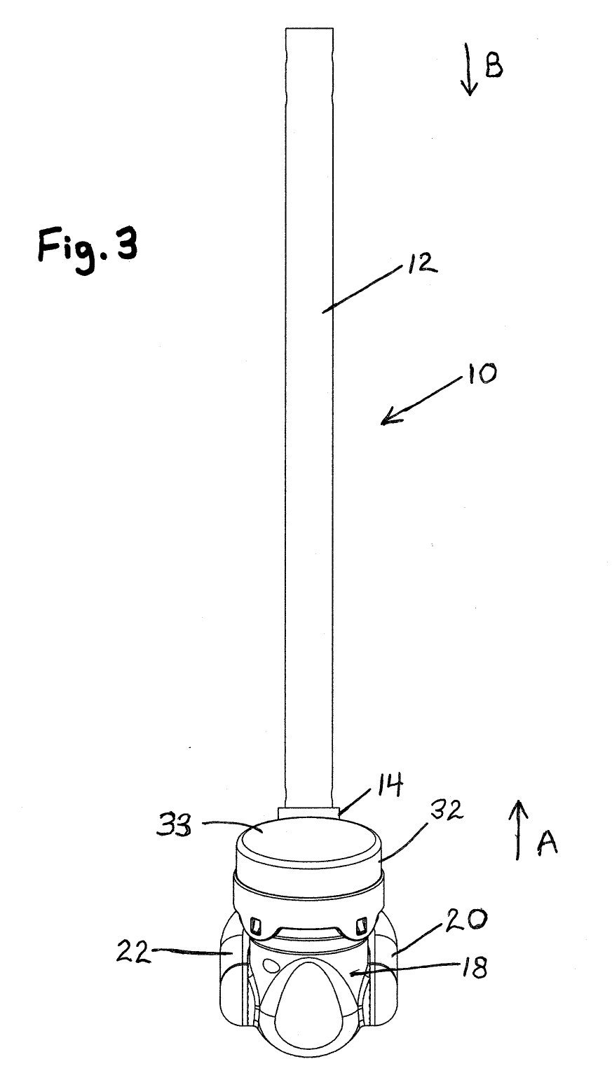

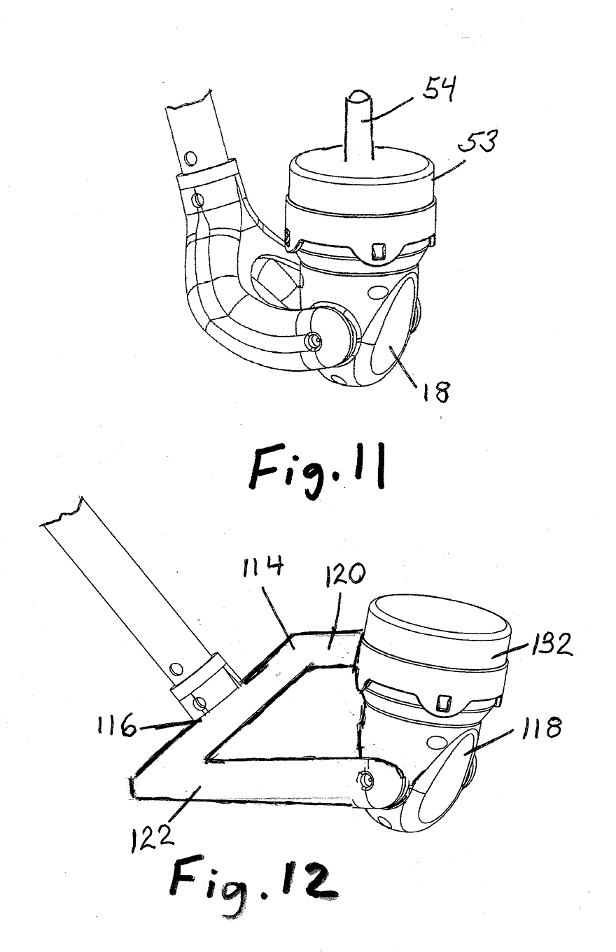

A frame 14 has a base portion 16 disposed adjacent to a first end of the handle 12. The frame 14 is used for attaching a mounting head 18 to the apparatus 10. The frame 14 has two spaced apart legs 20, 22 that originate at the base portion 16 of the frame. As shown in FIGS. 1, 2 and 4, each of the legs 20, 22 follows a curved path and the legs are spaced apart from one another a constant distance along their entire lengths, as shown in FIGS. 3, 5 and 6. Each of the legs 20, 22 has an end 24, 26 that is distal from the base portion 16 of the frame 14. However, it is understood that as shown in FIG. 12 each of the spaced apart legs 120, 122 that originate at a base portion 116 of the frame 114 may be straight with the legs spaced apart from one another a constant distance along their entire lengths. In either embodiment the frame may comprise any suitable material selected in accordance with good engineering practices, and may comprise the same material as the handle or a different material. The frame may be either a separate piece that is attached to the handle or made integral to the handle.

The mounting head 18; 118 is attached to the frame 14; 114 with a portion of the mounting head disposed between the legs 20, 22; 120, 122 of the frame. The mounting head 18 is fixed to the frame 14; 114 in a manner that allows the mounting head to rotate on an axis 30 that extends between the legs of the frame as shown in FIG. 1. In either embodiment, this feature may be provided for example by having the mounting head 18 fixed to the legs 20, 22; 120, 122 of the frame by a single axle 34 that extends through the mounting head and has a pair of ends with each of the ends of the axle being fixed to one of the legs of the frame. Alternatively, the mounting head may be fixed to the legs of the frame by a pair of axles 34, each of the axles having one end that is fixed to one of the legs of the frame and another end that is fixed to the mounting head. In either of these structures a ratchet mechanism 35 is preferably associated with each of the legs 20, 22; 120, 122 of the frame such that the axle(s) 34 may rotate to place the mounting head 18 in a desired orientation for treating either a top, bottom or side surface of a foot and retain the mounting head in a desired orientation until the orientation is changed by a person using the device. As used herein and in the claims the term “ratchet mechanism” is understood to have the common meaning of a mechanical device comprising g a toothed wheel or rack engaged with a pawl that permits it to move in only one direction.

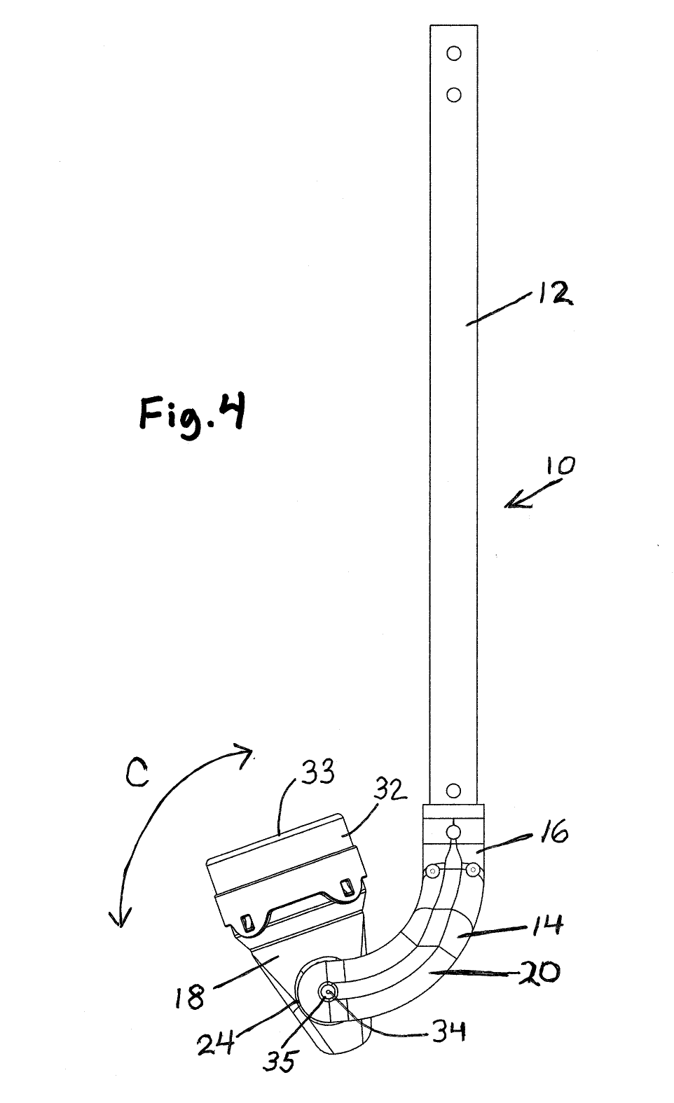

In either embodiment the mounting head can rotate along an arc C, as shown in FIG. 4, that allows a tool 32; 132 for treating a foot of a person fixed to the mounting head to be oriented with a working surface 33 of the tool oriented in any direction from the direction indicated by arrow A in FIG. 3 to the direction indicated by arrow B in FIG. 3. This is a great advantage of the apparatus of the present invention over the prior art because it allows a person with limited agility to treat the top, bottom, sides and heel of his foot. It is preferred that the fit between the mounting head 18 and the adjacent surfaces of the legs 20, 22; 120, 122 be relatively snug, that is to say a friction fit, that allows the mounting head to rotate with minimum effort by the user of the apparatus, while allowing the mounting head to remain in a desired orientation.

As used herein and in the claims the term “tool” is understood to have its' ordinary meaning of something used in order to perform a job or to achieve a goal. In FIGS. 1-7 and 12 the tool 32; 132 for treating a foot of a person is a pad for applying a lotion to the foot of a person. Preferably the pad is a foam pad. It is preferable that the tool 32 is fixed to the mounting head 18 in a removable and replaceable manner. An example of a means for attaching a tool to a mounting head in a removable and replaceable manner that is shown in the drawing is to use tabs 40 that extend through complementary openings 42 to secure the tool to the mounting head. However, it is understood that any suitable arrangement including mating threads and function al equivalents may be used in the practice of the invention.

Referring now to FIG. 7, there is shown an exploded view of a subassembly 100 of the mounting head 18 and the tool 32 for treating a foot of a person. When the tool 32 for treating a foot of a person is a pad for applying lotion the foot of a person it is desirable for the apparatus to be provided with a reservoir 38 for containing a lotion to be provided to the pad. While in FIG. 7 the reservoir 38 is located in the tool, it is understood that such a reservoir may be located in the mounting head if so desired.

If desired, as shown in FIG. 7, the mounting head 18 may be provided with an electro-mechanical means 39 for causing the tool attached to the mounting head to move. Alternatively the electro-mechanical means 39 may be configured to cause a reservoir 38 to provide lotion to the pad 32 for applying a lotion to a foot of a person. That is to say the reservoir is in communication with a lotion dispensing device for providing lotion directly to a person's foot or to the component of the device that is intended to come into contact with the foot of a person. The electro-mechanical means may comprise an energy source, such as a rechargeable battery or capacitor, and a suitable motor or vibrating mechanism. The motion imparted to the tool may be rotary motion, vibrating motion or oscillating motion, or any other desired motion selected by an engineer or designer to meet the needs of a user of the apparatus.

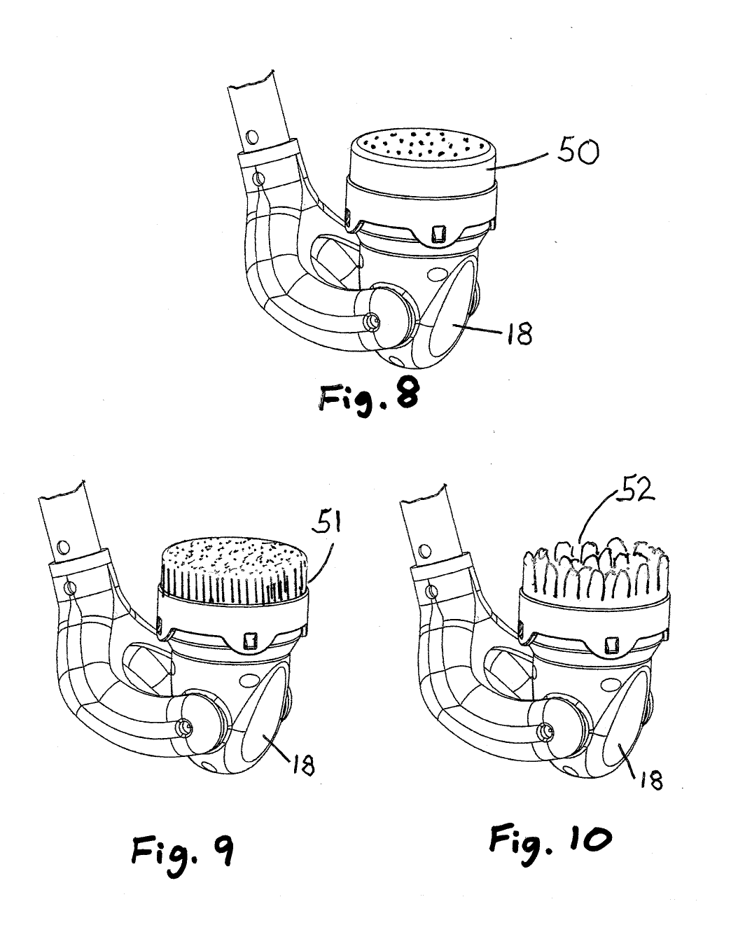

FIG. 8 shows an alternative tool 50 used with the apparatus of the present invention that comprises an abrasive member for treating a foot of a person. Such an abrasive member may be used for treating a callous located on a person's foot. The abrasive member may comprise, for example pumice. Inasmuch as in a preferred embodiment tools may be fixed to the mounting head, removed and then replaced by either a similar tool or a different tool, a person may both treat a callous by abrading away dead cells, and then applying a suitable lotion, or vice versa, using the apparatus of the present invention.

FIG. 9 shows an alternative tool 51 used with the apparatus of the present invention that comprises a brush that may be used for cleaning a foot of a person. Prior art foot cleaning brushes do not have the capacity to assume the wide range of orientations achievable by the rotation of the mounting head of the present invention as described above.

FIG. 10 shows an alternative tool 52 used with the apparatus of the present invention that comprises a massage head that may be used for massaging the foot of a person. The massage head 52 may be provided with flexible or stiff protrusions of any desirable size and stiffness. The massage head may be especially useful when the apparatus is provided with an electro-mechanical means for causing the tool to move, as described above. Prior art foot massaging devices do not have the capacity to assume the wide range of orientations achievable by the rotation of the mounting head of the present invention as described above.

FIG. 11 shows an alternative tool 53 used with the apparatus of the present invention that comprises a foam cylinder like member 54 that may be used for cleaning between a person's toes. This tool may be especially useful when the apparatus is provided with an electro-mechanical means, as described above, for causing the tool to move in with an up and down motion. Prior art foot cleaning devices do not have the capacity to assume the wide range of orientations achievable by the rotation of the mounting head of the present invention as described above.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense. It is also to be understood that the following claims are intended to cover all of the generic and specific features of the invention herein described, and all statements of the scope of the invention which, as a matter of language, might be said to fall there between.

What is claimed is:

1. An apparatus comprising:
a handle having a length that is greater than a width or diameter of the handle;
a frame having a base portion adjacent to a first end of the handle for attaching a mounting head to the apparatus, the frame having two spaced apart legs that originate at the base portion wherein the legs are spaced apart from one another a constant distance along their entire lengths, each of the legs having an end that is distal from the base portion of the frame;
the mounting head being attached to the frame with a portion of the mounting head disposed between the legs of the frame, the mounting head fixed to the frame in a manner that allows the mounting head to rotate on an axis that extends between the legs of the frame; and
a tool for treating a foot of a person fixed to the mounting head.

2. The apparatus according to claim 9 wherein the mounting head is provided with an electro-mechanical means for moving the mounting head.

3. The apparatus according to claim 1 wherein the mounting head is provided with an electro-mechanical means that vibrates, oscillates or rotates the tool attached to the mounting head.

4. The apparatus according to claim 1 wherein the mounting head is fixed to the legs of the frame by a single axle that extends through the mounting head and has a pair of ends with each of the ends of the axle being fixed to one of the legs of the frame using a ratchet mechanism.

5. The apparatus according to claim 1 wherein the mounting head is fixed to the legs of the frame by a pair of axles, each of the axles having one end that is fixed to one of the legs of the frame and another end that is fixed to the mounting head by a ratchet mechanism.

6. The apparatus according to claim 2 wherein the mounting head is fixed to the legs of the frame by a pair of axles, each of the axles having one end that is fixed to one of the legs of the frame and another end that is fixed to the mounting head by a ratchet mechanism.

7. The apparatus according to claim 3 wherein the mounting head is fixed to the legs of the frame by a pair of axles, each of the axles having one end that is fixed to one of the legs of the frame and another end that is fixed to the mounting head by a ratchet mechanism.

8. The apparatus according to claim 1 wherein the tool is fixed to the mounting head in a removable and replaceable manner.

9. The apparatus according to claim 1 wherein the tool for treating a foot of a person is a pad for applying a lotion to the foot of a person.

10. The apparatus according to claim 7 wherein the tool for treating a foot of a person is a pad for applying a lotion to the foot of a person.

11. The apparatus according to claim 9 wherein the apparatus further comprises a reservoir for containing a lotion, the reservoir being is in communication with a lotion dispensing device for providing lotion directly to a person's foot or to the pad.

12. The apparatus according to claim 10 wherein the apparatus further comprises a reservoir for containing a lotion, the reservoir being is in communication with a lotion dispensing device for providing lotion directly to a person's foot or to the pad.

13. The apparatus according to claim 1 wherein the tool for treating a foot of a person is an abrasive member.

14. The apparatus according to claim 1 wherein the tool for treating a foot of a person is a brush.

15. The apparatus according to claim 1 wherein the tool for treating a foot of a person is a massage head.

16. The apparatus according to claim 1 wherein the tool for treating a foot of a person includes a cylinder-like foam member for cleaning between a person's toes, and the mounting head is provided with an electro-mechanical means for moving the cylinder-like foam member to move up and down.

17. The apparatus according to claim 9 wherein the mounting head is provided with an electro-mechanical means that vibrates, oscillates or rotates the pad.

18. The apparatus according to claim 13 wherein the mounting head is provided with an electro-mechanical means that vibrates, oscillates or rotates the abrasive member.

19. The apparatus according to claim 14 wherein the mounting head is provided with an electro-mechanical means that vibrates, oscillates or rotates the brush.

20. The apparatus according to claim 15 wherein the mounting head is provided with an electro-mechanical means that vibrates, oscillates or rotates the massage head.
